(19) United States
(12) Patent Application Publication
(10) Pub. No.: US 2004/0214864 A1
(43) Pub. Date: Oct. 28, 2004

(54) Novel compounds

(76) Inventors: Peter Cage, Loughborough (GB); Barry Teobald, Loughborough (GB)

Correspondence Address: HATORI & CO., 8F YAMASHIRO BUILDING, NO. 1-15-16 MINAMIAOYAMA, MINATO-KU TOKYO JAPAN, JP

(21) Appl. No.: 10/240,657

(22) PCT Filed: Apr. 5, 2001
(86) PCT No.: PCT/SE01/00753

(30) Foreign Application Priority Data
Apr. 7, 2000 (GB) 0008464.0

(51) Int. Cl. C07D 417/04; C07D 249/12; C 07D 4 3/02; A61K 31/5377; A61K 31/4439; A61K 31/4196

(52) U.S. Cl. 514/341; 514/383; 546/270.4; 548/263.2

(57) ABSTRACT

The invention provides certain 1,2,4-triazole-3-thione compounds, processes for their preparation, pharmaceutical compositions containing them and their use in therapy.

The present invention relates to certain 1,2,4-triazole-3-thione compounds, processes for their preparation, pharmaceutical compositions containing them and their use in therapy.

WO 98/04135 discloses a class of substituted triazoles having the general formula

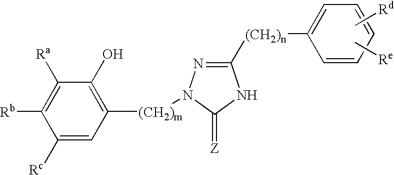

wherein Z is O or S; R^{a}, R^{b} and R^{c} each are independently selected from hydrogen, halogen, OH, CF₃, NO₂ or

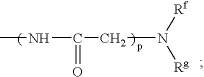

provided R^{c} is not hydrogen; and when R^{a} and R^{b} are hydrogen, R^{c} may be a heterocyclic moiety selected from the group consisting of imidazol-1-yl, morpholinomethyl, N-methylimidazol-2-yl and pyridin-2-yl; R^{d} and R^{c} each are independently selected from hydrogen, halogen, CF₃, NO₂ or imidazol-1-yl; m, n and p each are independently selected from an integer of 0 or 1; and R^{f} and R^{g} each are independently hydrogen; C₁-C₄ alkyl; or R^{f} and R^{g}, taken together with the nitrogen atom to which they are attached, is a heterocyclic moiety selected from the group consisting of N-methylpiperazine, morpholine, thiomorpholine, N-benzylpiperazine and imidazolinone. The substituted triazoles are said to act as potassium channel modulators, having application in the treatment of disorders such as ischaemia.

Further substituted triazole compounds having activity at chemokine receptors are disclosed in WO 00/12489.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. At the present time, the chemokine superfamily comprises three groups exhibiting characteristic structural motifs, the C—X—C, C—C and C—X₃—C families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR 11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

In accordance with the present invention, there is therefore provided a compound of general formula

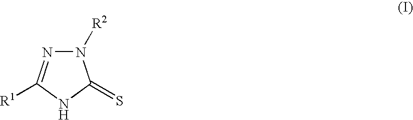

in which:

R¹ represents phenyl, naphthyl or a heterocyclic aromatic group containing at least one heteroatom selected from nitrogen, oxygen and sulphur;

R² represents a C₁₋₆ alkylaryl group;

where the aryl group of R² and/or the group R¹ is optionally substituted by one or more groups independently selected from halogen, NO₂, CN, C₁-C₆-alkyl itself optionally substituted by halogen, C(O)R⁸, OR⁸, SR⁸, NR⁹R¹⁰, C₃-C₇-cycloalkyl or phenyl and the aryl group of R² and/or the group R¹ is substituted by one or more groups of formula (CH₂)ₙX(CH₂)ₘY;

n and m are independently 0-4;

X is a bond, CO, NR³, SO₂, O or S;

Y is NR⁴COR⁵, CONR⁶R⁷, NR⁶R⁷ SO₂R⁸, OR⁸, SR⁸, NR⁸SO₂R⁸, SO₂NR⁶R⁷, COOR⁸ or tetrazol-5-yl;

R³, R⁴ and R⁵ are independently hydrogen, phenyl or C₁-C₆ alkyl which itself can be optionally substituted by halogen, NO₂, CN, C₁-C₆-alkyl (itself optionally substituted by halogen), C(O)R⁸, OR⁸, SR⁸, NR⁹R¹⁰, C₃-C₇-cycloalkyl or phenyl;

R⁶ and R⁷ are independently hydrogen, C₃-C₇ cycloalkyl or phenyl itself optionally substituted by one or more substituents selected from OR⁹, halogen, C₁-C₆ alkyl (itself optionally substituted by halogen), pyridinyl, imidazolyl-sulphonyl group, or a C₁-C₆ alkyl group (itself optionally substituted by one or more groups selected from halogen, OR⁸, COOR⁸ or NR⁹R¹), or R⁶ and R⁷ together with the nitrogen atom to which they are attached form a 3- to 7-membered heterocyclic ring optionally containing a further heteroatom selected from nitrogen, oxygen or sulphur and optionally substituted by one or more groups selected from R⁸ or NR⁹R¹⁰;

R⁸ is hydrogen, or C₁-C₆ alkyl or phenyl optionally substituted by halogen; and

R⁹ and R¹⁰ are independently hydrogen, phenyl or C₁₋₆ alkyl itself optionally substituted by halogen or phenyl,

and pharmaceutically acceptable salts and solvates thereof.

Examples of 3- to 7-membered heterocyclic rings optionally containing a further heteroatom selected from nitrogen, oxygen or sulphur and optionally substituted by one or more groups selected from R⁸ or NR⁹R¹⁰ include pyridine, pyrimidine, thiazole, oxazole, isoxazole, pyrole, imidazole, furan and thiophene.

Suitably R¹ represents phenyl, naphthyl or a heterocyclic aromatic group containing at least one heteroatom selected from nitrogen, oxygen and sulphur. Examples of suitable hetrocyclic aromatic groups includes furanyl, thienyl, pyridinyl or pyrimidinyl groups.

Preferably R¹ is phenyl substituted as defined above.

More preferably R¹ is phenyl substituted by:

halogen;

(CH₂)ₙX(CH₂)ₘY where n and m are 0, X is a bond and Y is COOR⁸ where R⁸ is hydrogen or C₁-C₆ alkyl or Y is SO₂NH₂ or Y is CONR⁶R⁷ where both of R⁶ or R⁷ are hydrogen C₁-C₆ alkyl or one of R⁶ or R⁷ is hydrogen and the other is alkyl optionally substituted by hydroxy and/or phenyl, NR⁹R¹⁰ or hydroxy and CO₂Me; or

(CH₂)ₙX(CH²)ₘY where n and m are 0, X is CO and Y is NHSO₂R⁸ where R⁸ is alkyl or phenyl

Most preferably R¹ is phenyl substituted by the substituents exemplified herein, namely: chloro, CO₂H, CO₂Me, CONH₂, CONMe₂, CONHCH₂CH₂CHMe₂, CONHCH(Me)CH(OH)Ph, CONHCH₂CH₂OH, 3-chloromethyl-1-piperazinylcarbonyl, CONHSO₂Me, CONHSO₂Ph, SO₂NH₂, CONHCH(CH₂OH)CO₂Me, and CONHCH₂C(Me₂)CH₂NMe,

Suitably R² represents a C₁₋₆ alkylaryl group. Preferably R² represents a benzyl group substituted by one or more groups independently selected from halogen or (CH₂)ₙX(CH₂)ₘY where n and m are 0, X is a bond and Y is COOR⁸ where R⁸ is hydrogen or alkyl or Y is CONR⁶R⁷ where one of R⁶ or R⁷ is hydrogen and the other is alkyl substituted by cyano or halogen. More preferably R² represents a benzyl group substituted by halogen, CO₂H, CO₂Me, CONHCH₂CN or CONHCH₂CH₂F. Even more preferably R² represents a benzyl group substituted by chloro, CO₂H, CO₂Me, CONHCH₂CN or CONHCH₂CH₂F.

Especially preferred compounds of the invention include:

4-[1-[(3-Chlorophenyl)methyl]-4,5-dihydro-5-thioxo-1H-1,2,4-triazol-3-yl]-benzoic acid)methyl]-4,5-dihydro-5-thioxo-1H-1,2,4-triazol-3-yl]-benzoic acid,

Methyl 4-[1-[(3-Chlorophenyl)methyl]-4,5-dihydro-5-thioxo-1H-1,2,4-triazol-3-yl]-benzoate,

4-[1-[(3-Chlorophenyl)methyl]-4,5-dihydro-5-thioxo-1H-1,2,4-triazol-3-yl]-benzamide,

4-[1-[(3-Chlorophenyl)methyl]-4,5-dihydro-5-thioxo-1H-1,2,4-triazol-3-yl]-N,N-dimethyl-benzamide,

4-[1-[(3-Chlorophenyl)methyl]-4,5-dihydro-5-thioxo-1H-1,2,4-triazol-3-yl]-N-(3-methylbutyl)-benzamide,

[1R, 2S]-4-[1-[(3-Chlorophenyl)methyl]-4,5-dihydro-5-thioxo-1H-1,2,4-triazol-3-yl]-N-(2-hydroxy-1-methyl-2-phenylethyl)-benzamide,

4-[1-[(3-Chlorophenyl)methyl]-4,5-dihydro-5-thioxo-1H-1,2,4-triazol-3-yl]-N-(2-hydroxyethyl)-benzamide,

2-[(3-Chlorophenyl)methyl]-5-[4-[[4-(3-chlorophenyl)-1-piperazinyl]carbonyl]phenyl]-2,4-dihydro-3H-1,2,4-triazole-3-thione,

N-[4-[1-[(3-Chlorophenyl)methyl]-4,5-dihydro-5-thioxo-1H-1,2,4-triazol-3-yl]benzoyl]-methanesulfonamide,

N-[4-[1-[(3-Chlorophenyl)methyl]-4,5-dihydro-5-thioxo-1H-1,2,4-triazol-3-yl]benzoyl]-benzenesulfonamide,

2-[1-[(3-Chlorophenyl)methyl]-4,5-dihydro-5-thioxo-1H-1,2,4-triazol-3-yl]-benzoic acid,

3-[1-[(3-Chlorophenyl)methyl]-4,5-dihydro-5-thioxo-1H-1,2,4-triazol-3-yl]-benzoic acid,

4-[1-[(3-chlorophenyl)methyl]-4,5-dihydro-5-thioxo-1H-1,2,4-triazol-3-yl]-benzenesulfonamide,

Methyl 2-[(3-)1-(3-chlorophenyl)methyl-1,2-dihydro-5-thioxo-1H[1,2,4]triazol-3-yl))phenylcarbonylamino]-3-hydroxypropanoate,

3-{1-[(3-Chlorophenyl)methyl]-1,2-dihydro-5-thioxo-1H[1,2,4]triazol-3-yl}-N-(2-methyl-2-dimethylaminomethylpropyl)benzamide,

3-{-[(3-Chlorophenyl)methyl]-1,2-dihydro-5-thioxo-1H[1,2,4]triazol-3-yl}-N,N-dimethylbenzamide,

{4-[5-(2-Chlorophenyl)-1,2-dihydro-3-thioxo-1H-[1,2,4]triazol-2-yl]methyl}-N-cyanomethylbenzamide,

{4-[5-(2-Chlorophenyl)-1,2-dihydro-3-thioxo-1H-[1,2,4]triazol-2-yl]methyl}-N-(2-fluoroethyl)benzamide,

and their pharmaceutically acceptable salts and solvates.

The present invention also provides a process for preparing a compound of formula (I) which comprises:

(a) reacting a compound of general formula (II), R¹—C(O)L, wherein L represents a leaving group (such as a halogen atom, e.g. chlorine) and R¹ is as defined in formula (I), with a compound of general formula

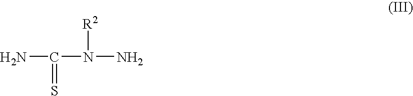

wherein R² is as defined in formula (I), followed by cyclisation; or

(b) reacting a compound of general formula

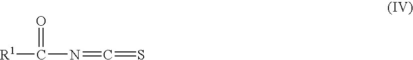

wherein R¹ is as defined in formula (I), with a compound of general formula

R²—NHNH₂ (V)

wherein R² is as defined in formula (I), followed by cyclisation; or

(c) reacting a compound of general formula

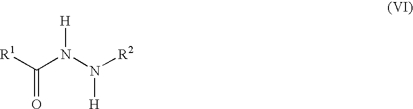

wherein R¹ and R² are as defined in formula (I), with ammonium thiocyanate, followed by cyclisation; or

(d) reacting a compound of general formula

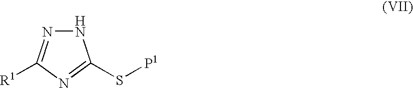

wherein P¹ represents a protecting group (e.g. methoxyethoxyrnethyl, triphenylmethyl or ethoxycarbonylethyl) and R¹ is as defined in formula (I), With a compound of general formula (VIII), R²—L¹, wherein. L¹ represents a leaving group (e.g. a halogen atom such as bromine, or an alcoholic group under Mitsunobu conditions) and R² is as defined in formula (I), followed by removal of the protecting group P¹ (e.g. by using suitable acidic or basic conditions);

and optionally after (a), (b), (c) or (d) forming a pharmaceutically acceptable salt or solvate of the compound of formula (I).

The process of the invention is conveniently carried out in an organic solvent such as dichloromethane, toluene, xylenes, triethylamine, pyridine or tetrahydrofuran at a temperature in the range, e.g. from 10 to 110° C. The cyclisation reaction may be effected under reflux conditions in the presence of sodium hydrogen carbonate, or sodium ethoxide in ethanol as described by H. Behringer et al., Liebigs Ann. Chem., 1975, 1264-1271.

Compounds of formula (II), (III), (IV), (V), (VI) and (VIII) are either commercially available, are well known in the literature or may be prepared easily using known techniques.

Compounds of formula (VII) may be prepared by a process analogous to that of step (a) above using the compound

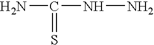

in place of the compound of formula (III).

It will be appreciated by those skilled in the art that in the processes described above the functional groups (e.g. hydroxyl groups) of intermediate compounds may need to be protected by protecting groups. The final stage in the preparation of the compounds of the invention may involve the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulphonate or p-toluenesulphonate, or an ammonium salt or an alkali metal salt such as a sodium or potassium salt.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention, particularly tautomers of general formula

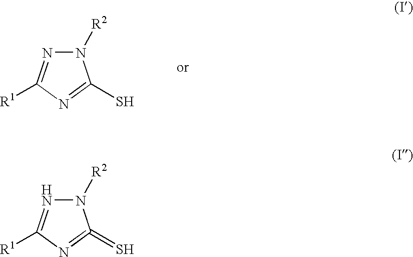

wherein R¹ and R² are as defined in formula (I) above.

The compounds of formula (I) have activity as pharmaceuticals, in particular as modulators of chemokine receptor (especially CXCR2) activity, and may be used in the treatment (therapeutic or prophylactic) of conditions/diseases in human and non-human animals which are exacerbated or caused by excessive or unregulated production of chemokines.

Examples of such conditions/diseases include:

(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;

(2) (bone and joints) rheumatoid arthritis, seronegative spondyloarthropathies (including ankylosing spondylitis, psoriatic arthritis and Reiter's disease), Behcet's disease, Sjogren's syndrome and systemic sclerosis;

(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;

(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;

(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal diorders, e.g. tropical spastic paraparesis, and stiff-man syndrome: paraneoplastic syndromes, e.g. cerebellar degeneration and encephalomyelitis; CNS trauma; migraine; and stroke.

(6) (other tissues and systemic disease) atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.

(7) (allograft rejection) acute and chronic following, for example, transplantation of kidney, heart, liver, lung, bone marrow, skin and cornea; and chronic graft versus host disease;

(8) Cancers, especially non-small cell lung cancer (NSCLC), malignant melanoma, prostate cancer and squamous sarcoma, and tumour metastasis;

(9) Diseases in which angiogenesis is associated with raised CXCR2 chemokine levels (e.g. NSCLC, diabetic retinopathy).

(10) Cystic fibrosis, re-perfusion injury in the heart, brain, peripheral limbs and other organs.

(11) Burn wounds & chronic skin ulcers

(12) Reproductive Diseases (e.g. Disorders of ovulation, menstruation and implantation, Pre-term labour, Endometriosis)

Thus, the present invention provides a compound of formula (I), or a pharmaceutically-acceptable salt or solvate thereof, as hereinbefore defined for use in therapy.

Preferably the compounds of the invention are used to treat diseases in which the chemokine receptor belongs to the CXC chemokine receptor subfamily, more preferably the target chemokine receptor is the CXCR2 receptor.

Particular conditions which can be treated with the compounds of the invention are psoriasis, diseases in which angiogenesis is associated with raised CXCR2 chemokine levels, and COPD. It is preferred that the compounds of the invention are used to treat psoriasis.

As a further aspect of the present invention, certain compounds of formula (I) may have utility as antagonists of the CX3CR1 receptor. Such compounds are expected to be particularly useful in the treatment of disorders within the central and peripheral nervous system and other conditions characterized by an activation of microglia and/or infiltration of leukocytes (e.g. stroke/ischemia and head trauma).

In a further aspect, the present invention provides the use of a compound of formula (I), or a pharmaceutically acceptable salt or solvate thereof, as hereinbefore defined in the manufacture of a medicament for use in therapy.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of chemokine receptor activity is beneficial, in particular modulation of the CXCR2 receptor.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CXCR2) receptor, which comprises administering to a patient a therapeutically effective amount of a compound of formula (I), or a pharmaceutically acceptable salt or solvate thereof, as hereinbefore defined.

The invention also provides a method of treating an inflammatory disease, especially psoriasis, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The invention further provides a-process for the preparation of a pharmaceutical composition of the invention which comprises mixing a compound of formula (I), or a pharmaceutically acceptable salt or solvate thereof, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compounds of the invention are administered orally.

The invention will now be further illustrated by reference to the following examples. In the examples the Nuclear Magnetic Resonance (NMR) spectra were measured on a Varian Unity Inova 300 or 400 MHz spectrometer and the Mass Spectrometry (MS) spectra measured on a Finnigan Mat SSQ7000 or Micromass Platform spectrometer. Where necessary, the reactions were performed under an inert atmosphere of either nitrogen or argon. Chromatography was generally performed using Matrex Silica 60® (35-70 micron) or Prolabo Silica gel 60® (35-70 micron) suitable for flash silica gel chromatography. High pressure liquid chromatography purification was performed using either a Waters Micromass LCZ with a Waters 600 pump controller, Waters 2487 detector and Gilson FC024 fraction collector or a Waters Delta Prep 4000. The abbreviations m.p. and DMSO used in the examples stand for melting point and dimethyl sulphoxide respectively.

### EXAMPLE 1

### 4-[1-[(3-Chlorophenyl)methyl]-4,5-dihydro-5-thioxo-1H-1,2,4-triazol-3-yl]-benzoic acid

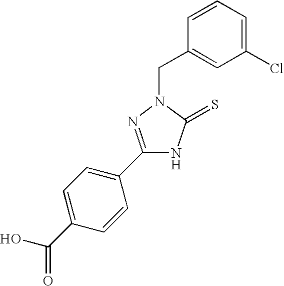

i) 2-(3-Chlorophenyl)methyl thiosemicarbazide

(3-Chlorophenyl)methylhydrazine hydrochloride (50.0 g) and ammonium thiocyanate (19.8 g) were heated at reflux in ethanol (200 ml) overnight. The hot suspension was filtered and the filtrate was allowed to cool and crystallize. The crystals were removed and dried to give the subtitle compound as colourless needles (27.6 g)

m.p.: 153-158° C.

MS: ESI (+ve) 216 (M+1, 100%)

¹H NMR: δ (DMSO) 7.65 (br, 2H), 7.42-7.27 (m, 4H), 5.22 (s, 2H), 4.75 (s, 2H)

ii) 4-[1-[(3-Chlorophenyl)methyl]-4,5-dihydro5-thioxo-1H-1,2,4-triazol-3-yl]-benzoic acid

Monomethyl terephthalate (1.67 g) and thionyl chloride (20 ml) were heated together at reflux for 1 hour, then concentrated in-vacuo to give a solid which was dissolved in pyridine (30 ml) and treated with the product from step (i) (2.00 g). The mixture was stirred for 3.5 hours then concentrated in-vacuo to remove the pyridine. The residue was suspended in 2M sodium hydroxide (30 ml) and heated at reflux for 5 hours. After allowing to cool overnight, the mixture was filtered to remove solids and washed with diethyl ether. The aqueous solution was acidified with 2M hydrochloric acid to give a precipitate which was removed by filtration and dried to give the title compound as a white powder (2.78 g).

m.p.: 302° C.

MS: ESI(−ve) 344 (M−1, 100%).

¹H NMR: δ (DMSO) 14.34 (br, 1H), 13.26 (br, 1H), 8.01-8.08 (m, 4H), 7.33-7.47 (m, 4H), 5.42 (s, 2H).

### EXAMPLE 2

### Methyl 4-[1-[(3-Chlorophenyl)methyl]-4,5-dihydro-5-thioxo-1H-1,2,4-triazol-3-yl]-benzoate

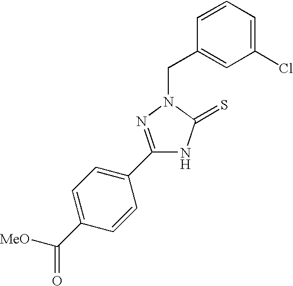

A suspension of the product from Example 1 step (ii) (0.20 g) in thionyl chloride (5 ml) was heated at reflux for 1 hour then concentrated in-vacuo. The residue was dissolved in tetrahydrofuran (5 ml) and methanol (1 ml) and stirred overnight. After concentration in-vacuo the crude product was purified by chromatography eluting with 3% methanol in dichloromethane followed by recrystallisation from ethyl acetate. Yield 0.023 g.

m.p.: 263.5-264.5° C.

MS: APCI(−ve) 358 (M−1, 100%).

¹H NMR: δ (DMSO) 14.35 (br, 1H), 8.04-8.07 (m, 4H), 7.35-7.46 (m, 4H), 5.41 (s, 2H), 3.88 (s, 3H).

### EXAMPLE 3

### 4-[1-[(3-Chlorophenyl)methyl]-4,5dihydro-5-thioxo-1H-1,2,4-triazol-3-yl]-benzamide

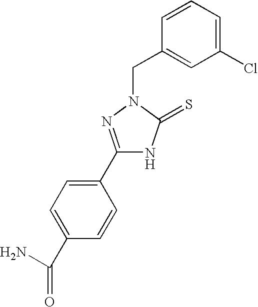

Prepared by the method of Example 2 using ‘880’ aqueous ammonia instead of methanol.

m.p.: 310-312° C.

MS: APCI(−ve) 343 (M−1, 100%).

¹H NMR: δ (DMSO) 14.27 (br, 1H), 8.09 (br, 1H), 7.97-8.02 (m, 4H), 7.52 (br, 1H), 7.34-7.46 (m, 4H), 5.41 (s, 2H).

### EXAMPLE 4

### 4-[1-[(3-Chlorophenyl)methyl]-4,5-dihydro-5-thioxo-1H-1,2,4-triazol-3-yl]-N,N-dimethyl-benzamide

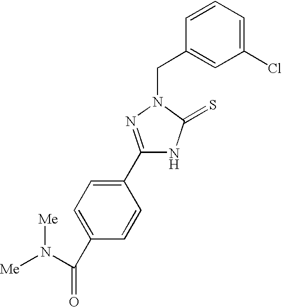

Prepared by the method of Example 2 using 2M dimethylamine solution in tetrahydrofuran instead of methanol.

m.p.: 302-304° C.

MS: APCI(−ve) 371 (M−1, 100%).

¹H NMR: δ (DMSO) 14.26 (br, 1H), 7.96 (d, 2H), 7.54 (d, 2H), 7.32-7.46 (m, 4H), 5.41 (s, 2H), 2.99 (br, 3H), 2.90 (br, 3H).

### EXAMPLE 5

### 4-[1-[(3-Chlorophenyl)methyl]-4,5-dihydro-5-thioxo-1H-1,2,4-triazol-3-yl]-N-(3-methylbutyl)-benzamide

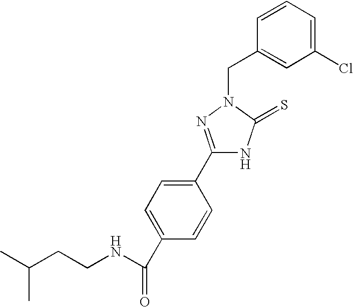

A suspension of the product from Example 1 step (ii) (0.25 g) in thionyl chloride (3.3 ml) was heated at reflux for 1 hour then concentrated in-vacuo. The residue was dissolved in tetrahydrofuran (5 ml) and treated with N,N-diisopropylethylamine (0.63 ml) followed by isoamylamine (0.17 ml). The mixture was stirred for 2 days, then concentrated in-vacuo. The crude product was purified by chromatography eluting with 2% methanol in dichloromethane, then by chromatography eluting with 33% ethyl acetate in isohexane, and finally by trituration with diethyl ether to give the title compound as a pale brown solid (0.023 g).

m.p.: 218-220° C.

MS: ESI(−ve) 413 (M−1, 100%).

¹H NMR: δ (DMSO) 14.26 (br, 1H), 8.54 (t, 1H), 7.93-8.00 (m, 4H), 7.32-7.45 (m, 4H), 5.41 (s, 2H), 3.25-3.32 (m, 2H), 1.58-1.64 (m, 1H), 1.39-1.46 (m, 2H), 0.90 (d, 6H).

### EXAMPLE 6

### [1R, 2S]-4-[1-[(3-Chlorophenyl)methyl]-4,5-dihydro-5-thioxo-1H-1,2,4-triazol-3-yl]-N-(2-hydroxy-1-methyl-2-phenylethyl)-benzamide

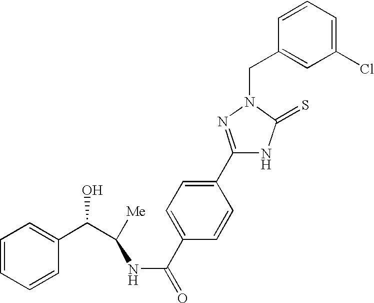

Prepared by the method of Example 7 using D-(+)-norephedrine instead of isoamylamine.

m.p.: 220-222° C.

MS: ESI(−ve) 477 (M−1, 100%).

¹H NMR: δ (DMSO) 14.25 (br, 1H), 8.34 (d, 1H), 7.88-7.97 (m, 4H), 7.17-7.45 (m, 9H), 5.41-5.45 (m, 3H), 4.70 (t, 1H), 4.12-4.18 (m, 1H), 1.11 (d, 3H).

### EXAMPLE 7

### 4-[1-[(3-Chlorophenyl)methyl]-4,5-dihydro-5-thioxo-1H-1,2,4-triazol-3-yl]-N-(2-hydroxyethyl)-benzamide

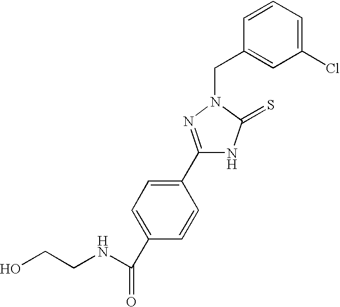

A suspension of the product from Example 1 step (ii) (0.20 g) in tetrahydrofuran (5 ml) was treated with 1,1′-carbonyldiimidazole (0.11 g) and stirred for 1.5 hours. Ethanolamine (0.070 ml) was added and the mixture was stirred overnight then concentrated in-vacuo. The crude product was purified by chromatography eluting with 5% methanol in dichloromethane and then by trituration with diethyl ether to give the title compound as a white powder (0.075 g).

m.p.: 188-190° C.

MS: ESI(+ve) 389 (M+1, 100%).

¹H NMR: δ (DMSO) 8.56 (t, 1H), 7.98 (br, 4H), 7.33-7.46 (m, 4H), 5.41 (s, 2H), 3.52 (t, 2H), 3.32-3.37 (m, 2H).

### EXAMPLE 8

### 2-[(3-Chlorophenyl)methyl]-5-[4-[[4-(3-chlorophenyl)-1-piperazinyl]carbonyl]phenyl]-2,4-dihydro-3H-1,2,4-triazole-3-thione

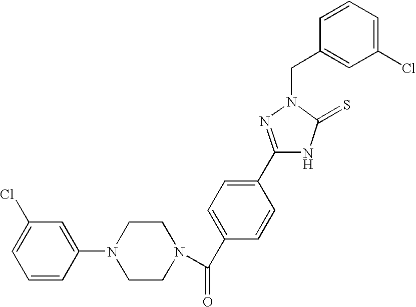

Prepared by the method of Example 7 using N,N-diisopropylethylamine and 1-(3-chloropbenyl)piperazine dihydrochloride instead of ethanolamine.

m.p.: 260-262° C.

MS: ESI(+ve) 524 (M+1, 100%).

¹H NMR: δ (DMSO at 90°) 13.93 (br, 1H), 7.97 (d, 2H), 7.56 (d, 2H), 7.33-7.44 (m, 4H), 7.20 (t, 1H), 6.92 (s, 1H), 6.87 (d, 1H), 6.79 (d, 1H), 5.39 (s, 2H), 3.61 (br, 4H), 3.24 (br, 4H).

### EXAMPLE 9

### N-[4-[1-[(3-Chlorophenyl)methyl]-4,5-dihydro-5-thioxo-1H-1,2,4-triazol-3-yl]benzoyl]-methanesulfonamide

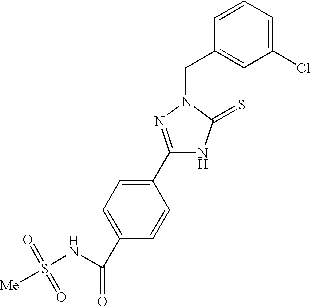

The product from Example 1 step (ii) (0.20 g), methanesulphonamide (0.062 g), N,N-dimethylaminopyridine (0.078 g) and 1-(3-dimethylaminopropyl)-3-ethylcarbodiimide hydrochloride (0. 125 g) were stirred together in dichloromethane (10 ml) for 6 days. The mixture was concentrated in-vacuo and the crude product was purified by chromatography eluting with 70% acetonitrile in dichloromethane followed by preparative reversed-phase HPLC (Waters Symmetry C₈ eluted with a gradient of 0.1% aqueous ammonium acetate buffer and acetonitrile). Lyophilisation gave the title compound as an amorphous solid (0.043 g).

MS: APCI(−ve) 421 (M−1, 100%).

¹H NMR: δ (DMSO) 8.04 (d, 2H), 7.93 (d, 2H), 7.33-7.46 (m, 4H), 5.41 (s, 2H), 3.05 (s, 3H).

### EXAMPLE 10

### N-[4-[1-[(3-Chlorophenyl)methyl]-4,5-dihydro-5-thioxo-1H-1,2,4-triazol-3-yl]benzoyl]-benzenesulfonamide

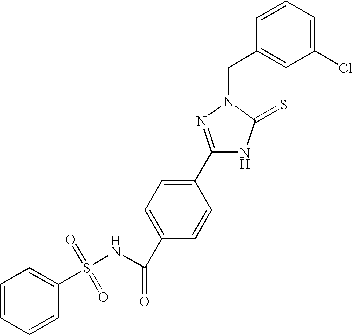

Prepared by the method of Example 9 using phenylsulphonamide instead of methanesulphonamide and heating the mixture at reflux for 2 days.

MS: APCI(−ve) 483 (M−1, 100%).

¹H NMR: δ (DMSO) 14.27 (br, 1H), 7.96-8.00 (m, 6H), 7.57-7.67 (m, 3H), 7.32-7.46 (m, 4H), 5.40 (s, 2H).

### EXAMPLE 11

### 2-[1-[(3-Chlorophenyl)methyl]-4,5-dihydro-5-thioxo-1H-1,2,4-triazol-3-yl]-benzoic acid

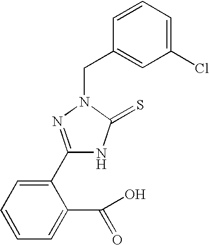

Prepared by the method of Example 1 step (ii) using methyl hydrogen phthalate instead of monomethylterephthalate.

m.p.: 234-238° C.

MS: APCI(−ve) 344 (M−1, 100%).

¹H NMR: δ (DMSO) 13.82 (br, 1H), 13.22 (br, 1H), 7.93-7.98 (m, 1H), 7.65-7.74 (m, 2H), 7.58-7.63 (m, 1H), 7.36-7.43 (m, 3H), 7.26-7.31 (m, 1H), 5.37 (s, 2H).

### EXAMPLE 12

### 3-[1-[(3-Chlorophenyl)methyl]-4,5-dihydro-5-thioxo-1H-1,2,4-triazol-3-yl]-benzoic acid

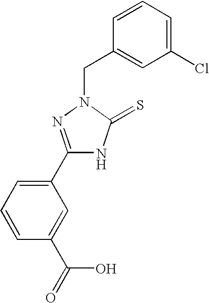

Prepared by the method of Example 1 step (ii) using monomethyl isophthalate instead of monomethyl terephthalate.

m.p.: 314-316° C.

MS: APCI(−ve) 344 (M−1, 100%).

¹H NMR: δ (DMSO) 14.33 (br, 1H), 13.29 (br, 1H), 8.52 (s, 1H), 8.14 (d, 1H), 8.07 (d, 1H), 7.66 (t, 1H), 7.34-7.47 (m, 4H), 5.41 (s, 2H).

### EXAMPLE 13

### 4-[1-[(3-chlorophenyl)methyl]-4,5dihydro-5-thioxo-1H-1,2,4-triazol-3-yl]-benzenesulfonamide

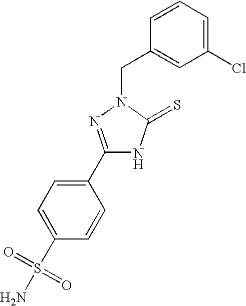

Prepared by the method of Example 1 step (ii) using 4-carboxybenzenesulphonamide instead of monomethyl terephthalate.

m.p.: 281-283° C.

MS: ESI(−ve) 379 (M−1, 100%).

¹H NMR: δ (DMSO) 14.34 (br, 1H), 8.07 (d, 2H), 7.93 (d, 2H), 7.33-7.50 (m, 6H), 5.41 (s, 2H).

### EXAMPLE 14

### Methyl 2-[(3-)1-(3-chlorophenyl)methyl-1,2-dihydro-5-thioxo-1H[1,2,4]triazol-3-yl))phenylcarbonylamino]-3-hydroxypropanoate

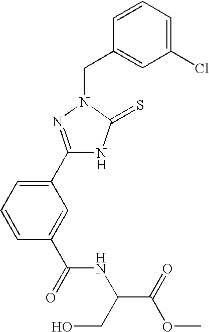

A solution of 3-[1-[(3-Chlorophenyl)methyl]-4,5-dihydro-5-thioxo-1H-1,2,4-triazol-3-yl]-benzoic acid (0.20 g) in dry tetrahydrofuran (5 ml) was stirred under nitrogen with 1,1′-carbonyldiimidazole (0.104 g) for 1 hour. DL-Serine methyl ester hydrochloride (0.109 g) and diethylisopropylamine (0.15 mL) were added and the mixture stirred 18 hours. The mixture was absorbed onto silica and purified by chromatography (dichloromethane: methanol, 97:3) to give the title compound (0.069 g).

m.p.: 188-196° C.

MS: APCI(+ve) 447 (M+1,100%).

¹H NMR: δ (DMSO) 14.28 (s, 1H), 8.73 (d, 1H), 8.41 (s, 1H), 8.07 (d, 1H), 8.02 (d, 1H), 7.65 (t, 1H), 7.44 (m,1H), 7.34 (m,2H), 5.42 (s, 2H),5.08 (t,1H), 4.57 (q,1H), 3.80 (t,2H), 3.66 (s, 3H).

### EXAMPLE 15

### 3-{1-[(3-Chlorophenyl)methyl]-1,2-dihydro-5thioxo-1H[1,2,4]triazol-3-yl}-N-(2-methyl-2-dimethylaminomethylpropyl)benzamide

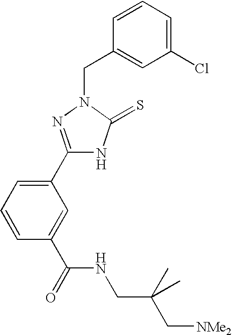

Prepared by the method of Example 15 using N,N,2,2-tetramethyl-1,3-propanediamine (0.222 mL) to give the title compound as a solid (0.046 g)

m.p.: 173-175° C.

MS: APCI(+ve) 458 (M+1,100%).

¹H NMR: δ (DMSO) 8.77 (t, 1H), 8.32 (s, 1H), 8.03 (d, 1H), 7.90 (d, 1H), 7.60 (t, 1H), 7.43 (s,1H), 7.36 (m, 2H), 7.32 (m, 1H), 5.40 (s, 2H), 3.21 (d,2H), 2.37 (s, 8H), 30.92 (s, 6H).

### EXAMPLE 16

### 3-{1-[(3-Chlorophenyl)methyl]-1,2-dihydro-5thioxo-1H[1,2,4]triazol-3-yl}-N,N-dimethylbenzamide

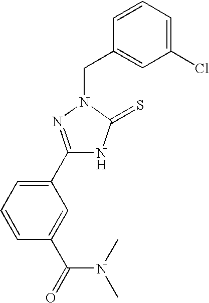

Prepared by the method of Example 15 using dimethylamine (1.4 mL of 2M in tetrahydrofuran) to give the title compound as a solid (0.039 g)

m.p.: 184-187° C.

MS: APCI(+ve) 373 (M+1,100%).

¹H NMR: δ (DMSO) 14.22 (s, 1H), 7.95 (m, 2H), 7.56 (m, 2H), 7.44 (s,1H), 7.35 (m, 3H), 5.40 (s, 2H), 3.00 (s, 3H), 2.92 (s, 3H).

### Example 17

### {4-[5-(2-Chlorophenyl)-1,2-dihydro-3-thioxo-1H-[1,2,4]triazol-2-yl]methyl}-N-cyanomethylbenzamide

(a) Methyl 3-[5-(2-chlorophenyl)-1,2-dihydro-3-thioxo-1H-[1,2,4]triazol-2-ylmethyl]benzoate

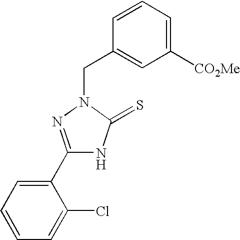

5-(2-Chlorophenyl)-3-triphenylmethylthio-1H-1,2,4-triazole (10 g), potassium carbonate (2.8 g) and methyl 3-bromomethylbenzoate (4.2 g) were stirred together in dry DMF (20 ml) is for 5 hrs. Water was added and the mixture extracted with dichloromethane. The extracts were washed with saturated sodium chloride solution then dried and filtered. Trifluoroacetic acid (2 ml) was added and the solution stood 10 mins. The solution was evaporated under reduced pressure. Purification was by chromatography eluting with 2% ethyl acetate in dichloromethane to give a solid (2.4 g). A sample (0.25 g) was further purified under the same conditions to give a solid which was triturated with ether to give a solid which was collected and dried. Yield 0.062 g

m.p.: 161-162° C.

MS: APCI(+ve): 360 (M+1), 328 (100%)

¹H NMR: δ (DMSO) 14.05 (bs, 1H), 7.98 (s, 1H), 7.91 (d, 1H), 7.64 (m, 3H), 7.53 (m, 3H), 5.47 (s, 2H), 3.85 (s, 3H).

(b) 3-[5-(2-chlorophenyl)-1,2-dihydro-3-thioxo-1H-[1,2,4]triazol-2-ylmethyl]benzoic acid

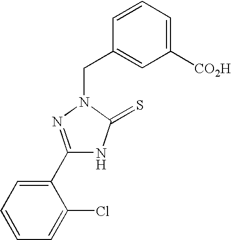

A solution of lithium hydroxide monohydrate (0.49 g) in water (50 ml) was added to a solution of methyl 3-[5-(2-chlorophenyl)-1,2-dihydro-3-thioxo-1H-[1,2,4]triazol-2-ylmethyl]benzoate (2.1 g) in methanol (150 ml) and the mixture stirred 18 hrs. The mixture was concentrated in vacuo and the residue taken up in water with sodium hydroxide solution (2 ml of 2M). The resulting solution was filtered and acidified with dilute hydrochloric acid. The solid which separated was collected by filtration, washed with water and air dried. Purification was by chromatography eluting with 5% methanol in dichloromethane to give a solid (1.1 g). The more pure fractions were collected, concentrated in vacuo to give a solid which was triturated with ether and collected then dried to give a solid (0.10 g).

m.p.: 257-261° C.

MS: APCI(+ve): 346 (M+1,100%)

¹H NMR: δ (DMSO) 14.04 (bs, 1H), 13.03 (bs, 1H), 7.96 (s, 1H), 7.89 (d, 1H), 7.60 (m, 3H), 7.50 (m, 2H), 5.46 (s, 2H).

### {4-[5-(2-Chlorophenyl)-1,2-dihydro-3-thioxo-1H-[1,2,4]triazol-2-yl]methyl}-N-cyanomethylbenzamide

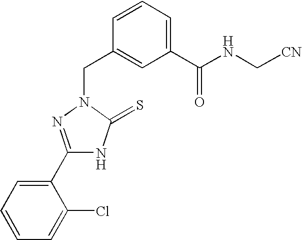

3-[5-(2-chlorophenyl)-1,2-dihydro-3-thioxo-1H-[1,2,4]triazol-2-ylmethyl]benzoic acid (0.346 g) was stirred in dichloromethane (3 ml) with oxalyl chloride (0.131 ml) and dry dimethylformamide (2 drops) for 3 hrs. The resulting solution was concentrated in vacuo then redissolved in dichloromethane (1 ml) and added to a suspension of aminoacetonitrile hydrochloride(0.111 g) and triethylamine (0.35 ml ) in dichloromethane(2 ml). The mixture was stirred 18 hrs. The mixture was diluted with dichloromethane and extracted into sodium bicarbonate solution. The aqueous was acidified with dilute hydrochloric acid and extracted into dichloromethane. The extracts were washed with saturated sodium chloride solution then dried and evaporated. Purification was by chromatography eluting with 30% ethyl acetate in dichloromethane to give a solid which was slurried with ether and collected to leave a solid (0.019 g).

m.p.: 178-182° C.

MS: APCI(+ve): 384 (M+1,100%)

¹H NMR: δ (DMSO) 14.04 (s, 1H), 9.24 (t, 1H), 7.88 (s, 1H), 7.80 (d, 1H), 7.67 (m, 2H), 7.50 (m, 4H), 5.45 (s, 2H), 4.31 (d, 2H).

### EXAMPLE 18

### {4-[5-(2-Chlorophenyl)-1,2-dihydro-3-thioxo-1H-[1,2,4]triazol-2-yl]methyl}-N-(2-fluoroethyl)benzamide

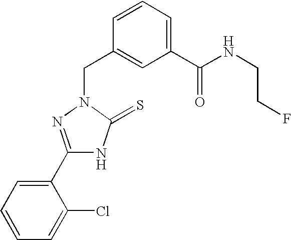

Prepared by the method of Example 20 using 2-fluoroethylamine hydrochloride (0.119 g) to give the title compound as a solid (0.053 g)

m.p.: 165-168° C.

MS: APCI(+ve) 391 (M+1,100%).

¹H NMR: δ (DMSO) 14.03 (s, 1H), 8.73 (t, 1H), 7.88 (s,1H), 7.80 (d, 1H), 7.66 (m, 2H), 7.58 (td, 1H), 7.50 (m, 4H), 5.44 (s, 2H), 4.61 (t, 1H), 4.45 (t,1H), 3.60 (q, 1H), 3.51 (q, 1H).

Pharmacological Data

Ligand Binding Assay

[¹²⁵I]IL-8 (human, recombinant) was purchased from Amersham, U.K. with a specific activity of 2,000 Ci/mmol. All other chemicals were of analytical grade. High levels of hrCXCR2 were expressed in HEK 293 cells (human embryo kidney 293 cells ECACC No. 85120602) (Lee et al. (1992) J Biol. Chem. 267 pp16283-16291). hrCXCR2 cDNA was amplified and cloned from human neutrophil mRNA. The DNA was cloned into PCRScript (Stratagene) and clones were identified using DNA. The coding sequence was sub-cloned into the eukaryotic expression vector RcCMV (Invitrogen). Plasmid DNA was prepared is using Quiagen Megaprep 2500 and transfected into HEK 293 cells using Lipofectamine reagent (Gibco BRL). Cells of the highest expressing clone were harvested in phosphate-buffered saline containing 0.2% (w/v) ethylenediaminetetraacetic acid (EDTA) and centrifuged (200 g, 5 min.). The cell pellet was resuspended in ice cold homogenisation buffer [10 mM HEPES (pH 7.4), 1 mM dithiothreitol, 1 mM EDTA and a panel of protease inhibitors (1 mM phenyl methyl sulphonyl fluoride, 2 μg/ml soybean trypsin inhibitor, 3 mM benzamidine, 0.5 μg/ml leupeptin and 100 μg/ml bacitracin)] and the cells left to swell for 10 minutes. The cell preparation was disrupted using a hand held glass mortar/PTFE pestle homogeniser and cell membranes harvested by centrifugation (45 minutes, 100,000 g, 4° C.). The membrane preparation was stored at −70° C. in homogenisation buffer supplemented with Tyrode's salt solution (137 mM NaCl, 2.7 mM KCl, 0.4 mM NaH₂PO₄), 0.1% (w/v) gelatin and 10% (v/v) glycerol.

All assays were performed in a 96-well MultiScreen 0.45 μm filtration plates (Millipore, U.K.). Each assay contained ˜33 pM [¹²⁵I]IL-8 and membranes (equivalent to ˜80,000 cells) in assay buffer [Tyrode's salt solution supplemented with 10 mM HEPES (pH 7.4), 1.8 mM CaCl₂, 1 mM MgCl₂, 0.5 mg/ml bacitracin and 0.1% (w/v) gelatin]. In addition, a compound of formula (I) according to the Examples was pre-dissolved in DMSO and added to reach a final concentration of 1% (v/v) DMSO. The assay was initiated with the addition of membranes and after 1.5 hours at room temperature the membranes were harvested by filtration using a Millipore MultiScreen vacuum manifold and washed twice with assay buffer (without bacitracin). The backing plate was removed from the MultiScreen plate assembly, the filters dried at room temperature, punched out and then counted on a Cobra γ-counter.

The compounds of formula (I) according to the Examples were found to have IC₅₀ values of less than (<) 10 μM.

Intracellular Calcium Mobilisation Assay

Human neutrophils were prepared from EDTA-treated peripheral blood, as previously described (Baly et al. (1997) Methods in Enzymology 287 pp70-72), in storage buffer [Tyrode's salt solution (137 mM NaCl, 2.7 mM KCl, 0.4 mM NaH₂PO₄) supplemented with 5.7 mM glucose and 10 mM HEPES (pH 7.4)].

The chemokine GROα (human, recombinant) was purchased from R&D Systems (Abingdon, U.K.). All other chemicals were of analytical grade. Changes in intracellular free calcium were measured fluorometrically by loading neutrophils with the calcium sensitive fluorescent dye, fluo-3, as described previously (Merritt et al. (1990) Biochem. J. 269, pp513-519). Cells were loaded for 1 hour at 37° C. in loading buffer (storage buffer with 0.1% (w/v) gelatin) containing 5 μM fluo-3 AM ester, washed with loading buffer and then resuspended in Tyrode's salt solution supplemented with 5.7 mM glucose, 0.1% (w/v) bovine serum albumin (BSA), 1.8 mM CaCl₂ and 1 mM MgCl₂. The cells were pipetted into black walled, clear bottom, 96 well micro plates (Costar, Boston, U.S.A.) and centrifuged (200 g, 5 minutes, room temperature).

A compound of formula (I) according to the Examples was pre-dissolved in DMSO and added to a final concentration of 0.1% (v/v) DMSO. Assays were initiated by the addition of an A₅₀ concentration of GROα and the transient increase in fluo-3 fluorescence (λ_{Ex}=490 nm and λ_{Em}=520 nm) monitored using a FLIPR (Fluorometric Imaging Plate Reader, Molecular Devices, Sunnyvale, U.S.A.).

The compounds of formula (I) according to the Examples were tested and found to be antagonists of the CXCR2 receptor in human neutrophils.

1. A compound of general formula 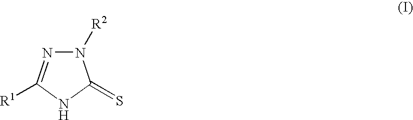
in which:
R¹ represents phenyl, naphthyl or a heterocyclic aromatic group containing at least one heteroatom selected from nitrogen, oxygen and sulphur;
R² represents a C₁₋₆ alkylaryl group;
where the aryl group of R² and/or the group R¹ is optionally substituted by one or more groups independently selected from halogen, NO₂, CN, C₁-C₆-alkyl itself optionally substituted by halogen, C(O)R⁸, OR⁸SR⁸, NR⁹R¹⁰, C₃-C₇-cycloalkyl or phenyl and the aryl group of R² and/or the group R¹ is substituted by one or more groups of formula (CH₂)ₙX(CH₂)ₘY;
n and m are independently 0-4;
X is a bond, CO, NR³, SO₂, O or S;
Y is NR⁴COR⁵, CONR⁶R⁷, NR⁶R⁷ SO₂R⁸, OR⁸, SR⁸, NR⁸SO₂R⁸, SO₂NR⁶R⁷, COOR⁸ or tetrazol-5-yl;
R³, R⁴ and R⁵ are independently hydrogen, phenyl or C₁-C₆ alkyl which itself can be optionally substituted by halogen, NO₂, CN, C₁-C₆-alkyl (itself optionally substituted by halogen), C(O)R⁸, OR⁸, SR⁸, NR⁹R¹⁰, C₃-C₇-cycloalkyl or phenyl;
R⁶ and R⁷ are independently hydrogen, C₃-C₇ cycloalkyl or phenyl itself optionally substituted by one or more substituents selected from OR⁹, halogen, C₁-C₆ alkyl (itself optionally substituted by halogen), pyridinyl, imidazolyl-sulphonyl group, or a C₁-C₆ alkyl group (itself optionally substituted by one or more groups selected from halogen, OR⁸, COOR⁸ or NR⁹R¹⁰), or R⁶ and R⁷ together with the nitrogen atom to which they are attached form a 3- to 7-membered heterocyclic ring optionally containing a further heteroatom selected from nitrogen, oxygen or sulphur and optionally substituted by one or more groups selected from R⁸ or NR⁹R¹⁰;
R⁸ is hydrogen, or C₁-C₆ alkyl or phenyl optionally substituted by halogen; and
R⁹ and R¹⁰ are independently hydrogen, phenyl or C₁₋₆ alkyl itself optionally substituted by halogen or phenyl, and pharmaceutically acceptable salts and solvates thereof.

2. A compound according to claim 1, wherein R¹ is phenyl substituted as defined in claim 1

3. A compound according to claim 1 or 2 wherein R¹ is phenyl substituted by:
halogen;
(CH₂)ₙX(CH₂)ₘY where n and m are 0, X is a bond and Y is COOR⁸ where R⁸ is hydrogen or C₁-C₆ alkyl or Y is SO₂NH₂ or Y is CONR⁶R⁷ where both of R⁶ or R⁷ are hydrogen or C₁-C₆ alkyl or one of R⁶ or R⁷ is hydrogen and the other is alkyl optionally substituted by hydroxy and/or phenyl, NR⁹R¹⁰ or hydroxy and CO₂Me; or
(CH₂)ₙX(CH₂)ₘY where n and m are 0, X is CO and Y is NHSO₂R⁸ where R⁸ is alkyl or phenyl.

4. A compound according to claim 1 or 2 wherein R¹ is phenyl substituted by chloro, CO₂H, CO₂Me, CONH₂, CONMe2, CONHCH₂CH₂CHMe₂, CONHCH(Me)CH(OH)Ph, CONHCH₂CH₂OH, 3-chloromethyl-1-piperazinylcarbonyl, CONHSO₂Me, CONHSO₂Ph, SO₂NH₂, CONHCH(CH₂OH)CO₂Me or CONHCH₂C(Me₂)CH₂NMe₂.

5. A compound according to any one of claims 1 to 4 wherein R² represents a benzyl group substituted by one or more groups independently selected from halogen or (CH₂)ₙX(CH₂)ₘY where n and m are 0, X is a bond and Y is COOR⁸ where R⁸ is hydrogen or alkyl or Y is CONR⁶R⁷ where one of R⁶ or R⁷ is hydrogen and the other is alkyl substituted by cyano or halogen.

6. A compound according to any one of claims 1 to 4 wherein R² represents a benzyl group substituted by halogen, COOH, COOMe, CONHCH₂CN or CONHCH₂CH₂F.

7. A compound according to any one of claims 1 to 6 selected from:
4-[1-[(3-Chlorophenyl)methyl]-4,5-dihydro-5-thioxo 1H-1,2,4-triazol-3-yl]-benzoic acid)methyl]-4,5-dihydro-5-thioxo-1H-1,2,4-triazol-3-yl]-benzoic acid,
Methyl 4-[1-[(3-Chlorophenyl)methyl]-4,5-dihydro-5-thioxo-1H-1,2,4-triazol-3-yl]-benzoate,
4-[1-[(3-Chlorophenyl)methyl]-4,5-dihydro-5-thioxo-1H-1,2,4-triazol-3-yl]-benzamide,
4-[1-[(3-Chlorophenyl)methyl]-4,5-dihydro-5-thioxo-1H-1,2,4-triazol-3-yl]-N,N-dimethyl-benzamide,
4-[1-[(3-Chlorophenyl)methyl]-4,5-dihydro-5-thioxo-1H-1,2,4-triazol-3-yl]-N-(3-methylbutyl)-benzamide,
[1R, 2S]-4-[1-[(3-Chlorophenyl)methyl]-4,5-dihydro-5-thioxo-1H-1,2,4-triazol-3-yl]-N-(2-hydroxy-1-methyl-2-phenylethyl)-benzamide,
4-[1-[(3-Chlorophenyl)methyl]-4,5-dihydro-5-thioxo-1H-1,2,4-triazol-3-yl]-N-(2-hydroxyethyl)-benzamide,
2-[(3-Chlorophenyl)methyl]-5-[4-[[4-(3-chlorophenyl)-1-piperazinyl]carbonyl]phenyl]-2,4-dihydro-3H-1,2,4-triazole-3-thione,
N-[4-[1-[(3-Chlorophenyl)methyl]-4,5-dihydro-5-thioxo-1H-1,2,4-triazol-3-yl]benzoyl]-methanesulfonamide,
N-[4-[1-[(3-Chlorophenyl)methyl]-4,5-dihydro-5-thioxo-1H-1,2,4-triazol-3-yl]benzoyl]-benzenesulfonamide,
2-[1-[(3-Chlorophenyl)methyl]-4,5-dihydro-5-thioxo-1H-1,2,4-triazol-3-yl]-benzoic acid,
3-[1-[(3-Chlorophenyl)methyl]-4,5-dihydro-5-thioxo-1H-1,2,4-triazol-3-yl]-benzoic acid,
4-[1-[(3-Chlorophenyl)methyl]-4,5-dihydro-5-thioxo-1H-1,2,4-triazol-3-yl]-benzenesulfonamide,
Methyl 2-[(3-)1-(3-chlorophenyl)methyl-1,2-dihydro-5-thioxo-1H[1,2,4]triazol-3-yl))phenylcarbonylamino]-3-hydroxypropanoate,
3-{1-[(3-Chlorophenyl)methyl]-1,2-dihydro-5-thioxo-1H[1,2,4]triazol-3-yl}-N-(2-methyl-2-dimethylaminomethylpropyl)benzamide,
3-{1-[(3-Chlorophenyl)methyl]-1,2-dihydro-5-thioxo-1H[1,2,4]triazol-3-yl }-N,N-dimethylbenzamide,
{4-[5-(2-Chlorophenyl)-1,2-dihydro-3-thioxo-1H-[1,2,4]triazol-2-yl]methyl}-N-cyanomethylbenzamide,
{4-[5-(2-Chlorophenyl)-1,2-dihydro-3-thioxo-1H-[1,2,4]triazol-2-yl]methyl}-N-(2-fluoroethyl)benzamide,
and their pharmaceutically acceptable salts and solvates.

8. A process for preparing a compound of formula (I) as defined in claim 1 which comprises:
(a) reacting a compound of general formula (II), R¹—C(O)L, wherein L represents a leaving group and R¹ is as defined in formula (I), with a compound of general formula 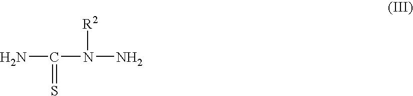
wherein R² is as defined in formula (I), followed by cyclisation; or
(b) reacting a compound of general formula 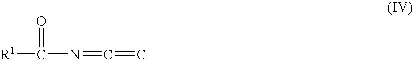
wherein R¹ is as defined in formula (I), with a compound of general formula
R²—NHNH₂ (V)
wherein R² is as defined in formula (I), followed by cyclisation; or
(c) reacting a compound of general formula 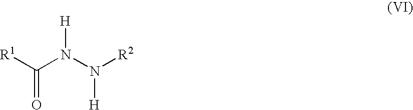
wherein R¹ and R² are as defined in formula (I), with ammonium thiocyanate, followed by cyclisation; or
(d) reacting a compound of general formula 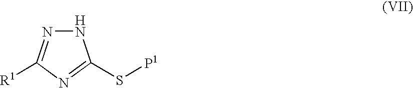
wherein P¹ represents a protecting group and R¹ is as defined in formula (I), with a compound of general formula (VIII), R²—L¹, wherein L¹ represents a leaving group and R² is as defined in formula (I), followed by removal of the protecting group P¹;
and optionally after (a), (b), (c) or (d) forming a pharmaceutically acceptable salt or solvate of the compound of formula (I).

9. A pharmaceutical composition comprising a compound of formula (I), or a pharmaceutically acceptable salt or solvate thereof, as claimed in any one of claims 1 to 7 in association with a pharmaceutically acceptable adjuvant, diluent or carrier.

10. A process for the preparation of a pharmaceutical composition as claimed in claim 7 which comprises mixing a compound of formula (I), or a pharmaceutically acceptable salt or solvate thereof, as claimed in any one of claims 1 to 7 with a pharmaceutically acceptable adjuvant, diluent or carrier.

11. A compound of formula (I), or a pharmaceutically-acceptable salt or solvate thereof, as claimed in any one of claims 1 to 7 for use in therapy.

12. Use of a compound of formula (I), or a pharmaceutically acceptable salt or solvate thereof, as claimed in any one of claims 1 to 7 in the manufacture of a medicament for use in therapy.

13. A method of treating a chemokine mediated disease wherein the chemokine binds to a one or more chemokine receptors, which comprises administering to a patient a therapeutically effective amount of a compound of formula (I), or a pharmaceutically acceptable salt or solvate thereof, as claimed in any one of claims 1 to 7.

14. A method according to claim 13 in which the chemokine receptor belongs to the CXC chemokine receptor subfamily.

15. A method according to claim 13 or 14 in which the chemokine receptor is the CXCR2 receptor.

16. A method according to claims 13 to 15 wherein the disease is psoriasis, a disease in which angiogenesis is associated with raised CXCR2 chemokine levels, or COPD.

17. A method according to claim 16, wherein the disease is psoriasis.
